(19) United States
(12) Patent Application Publication
(10) Pub. No.: US 2018/0369093 A1
(43) Pub. Date: Dec. 27, 2018

(54) Gel Nail Polish System and Method of Applying Gel Nail Polish

(71) Applicant: Vu V. Tran, RICHARDSON, TX (US)

(72) Inventor: Vu V. Tran, RICHARDSON, TX (US)

(21) Appl. No.: 16/008,715

(22) Filed: Jun. 14, 2018

Related U.S. Application Data
(60) Provisional application No. 62/523,026, filed on Jun. 21, 2017.

(51) Int. Cl. A61K 8/37; A61Q 3/02; A61K 8/04; A61K 8/19; A61K 8/35; A61K 8/40

(52) U.S. Cl. CPC A61K 8/37; A61Q 3/02; A61K 8/042; A45D 2200/25; A61K 8/35; A61K 8/40; A61K 8/19

(57) ABSTRACT

A gel nail polish system and method of applying the same. A gel nail polish system including a foundation layer that removes oil and dirt buildup on a nail, a base layer to provide an even and uniform surface across the nail, a color layer having a pigment, wherein the color layer reacts with the previous layers to harden the gel nail polish, and a top coat that cures to protect the previous layers from damage. The method for applying the gel nail polish includes applying one or more layers of the foundation layer, waiting for the foundation layer to dry, applying one or more layers of the base layer, waiting for the base layer to harden, applying a top coat over the nail, and waiting for the top coat to cure.

### CROSS REFERENCE TO RELATED APPLICATIONS

This application claims the benefit of U.S. Provisional Application No. 62/523,026 filed on Jun. 21, 2017. The above identified patent application is herein incorporated by reference in its entirety to provide continuity of disclosure.

### BACKGROUND OF THE INVENTION

The present invention relates to gel nail polish systems. Specifically, it relates to gel nail polish systems that chemically react to cure without the use of UV light.

Many people enjoy having manicures and decorating their fingernails to fit with their personal style or accessorize with a specific outfit. Traditional nail polish can be used, however traditional nail polish chips easily, thereby ruining the appearance of the nail. In order to circumvent this, many people choose to go to a salon to have a gel nail polish applied, as gel nail polishes are generally more resilient and remain intact for longer periods of time. Gel nail polishes involve a curing step that cross-links polymer chains, providing increased durability, toughness, and hardness. However, in order to apply a traditional gel nail polish, the gel nail polish must be cured via ultraviolet light, which can be harmful and damaging to the nail bed and surrounding skin in certain circumstances. In these cases, ultraviolet light causes the polymer chains therein to cross-link providing increased durability. Additionally, going to a salon regularly can be expensive and result in a large time investment. Therefore, a self-curing gel nail polish that an amateur can use on their own is desired.

In light of the devices disclosed in the known art, it is submitted that the present invention substantially diverges in design elements from the known art and consequently it is clear that there is a need in the art for an improvement to existing gel nail polish systems. In this regard, the instant invention substantially fulfills these needs.

### SUMMARY OF THE INVENTION

In view of the foregoing disadvantages inherent in the known types of gel nail polish systems now present in the known art, the present invention provides a gel nail polish system wherein the same can be utilized for providing convenience for the user when applying gel nail polish without the use of a UV light source.

The present system comprises a foundation layer comprising a solution of ethyl acetate, ammonium hydroxide, and a ketone, wherein the foundation layer is configured to remove oil and dirt buildup on the nail and evaporate over time leaving a deposit of ketone on the nail. The gel nail polish system further includes a base layer comprising a solution of ethyl cyanoacrylate, wherein the base layer is configured to provide a uniform surface across a nail. The gel nail polish system further includes a color layer comprising a solution of ethyl methacrylate, a ketone, a plasticizer, and a pigment, wherein the ketone is configured to penetrate through the base layer to react with the foundation layer to harden each applied layer. The gel nail polish further includes a top coat comprising a solution of ethyl cyanoacrylate, wherein the top coat is configured to harden to protect the color, base, and foundation layers from damage. Each of the foundation layer, base layer, color layer, and top coat are in solution with a solvent. In some embodiments, the foundation layer comprises 80% ethyl acetate, 15% ammonium hydroxide, and 5% ketone. In another embodiment, the base layer comprises 50-100% ethyl cyanoacrylate, wherein the balance comprises the solvent. In other embodiments, the pigment comprises 5% of the color layer.

The method of applying a gel nail polish system comprises applying at least one foundation layer comprising a solution of ethyl acetate, ammonium hydroxide, and a ketone to remove dirt and oil buildup on a nail, pausing until the foundation layer has fully evaporated, leaving a coating of ketone on the nail. The method further includes applying at least one base layer comprising a solution of ethyl cyanoacrylate, wherein the base layer is configured to smooth an outer surface of the nail such that the nail comprises a uniform outer surface and pausing until the base layer has fully evaporated to allow the ketone to penetrate each of the layers to react and harden each of the applied layers. The method further includes applying at least one top coat comprising a solution of ethyl cyanoacrylate and pausing to allow the top coat to cure the protect each of the previously applied layers. In some embodiments, the method further comprises washing the nail surface before applying the foundation layer. In another embodiment, the method includes applying a color layer before applying the top coat, wherein the color layer comprises a solution of ethyl methacrylate, a ketone, a plasticizer, and a pigment. In other embodiments, the method further comprises pausing until the color layer has evaporated, such that the ketone therein has reacted with the previously applied layers to harden. In yet another embodiment, the method further comprises polishing the nail after the top coat has fully cured.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a flowchart of the method of applying an embodiment of the gel nail polish system.

### DETAILED DESCRIPTION OF THE INVENTION

Reference is made herein to the attached drawings. Like reference numerals are used throughout the drawings to depict like or similar elements of the gel nail polish system. The figures are intended for representative purposes only and should not be considered to be limiting in any respect.

The gel nail polish system comprises a foundation layer, a base layer, a color layer, and a top coat. Each of the foundation layer, base layer, color layer, and top coat is configured to chemically react with each of the remaining layers, such that as each layer is applied, a chemical reaction occurs to harden and cure each layer. In this way, as each layer is applied, the gel nail polish system creates a durable and resilient coating on a nail, allowing the user can decide which of the layers the user wishes to apply. For example, should a user prefer to have a colorless nail polish, the user can apply the foundation layer, the base layer, and the top coat, and the gel nail polish system will still cure absent the color layer, effectively producing a durable gel nail polish without the application of a color layer. As the gel nail polish system of the present invention relies upon chemical reactions to set and cure, a user requires no ultraviolet light treatment to cure the gel nail polish. This allows a user to apply the gel nail polish without expensive UV light equipment. Additionally, as the gel nail polish is not reactive to UV light, the gel nail polish can be stored in transparent or translucent storage containers without affecting the gel nail polish, thereby allowing a user to visually compare various tones or colors of gel nail polish.

Each of the foundation, base, color, and top coat layers is in solution with a solvent. In a preferred embodiment, the solvent comprises a ketone, such that the ketone can be used as a polymer precursor when reacted with elements found in subsequent layers. However, alternate solvents that do not interfere with the intermediate reactions between individual layers are contemplated, such as, but not limited to, esters, alcohols, and aromatics. Specific examples contemplated include a mixture of one or more of butyl acetate, ethyl acetate, propyl acetate, dimethyl-p-toluidine, N-dimethyl-p-toluidine, butyl alcohol, isopropyl alcohol, ethanol, amyl acetate, or acetone. Further solvents capable of acting as polymer precursors and having similar properties are also contemplated. For brevity, the remainder of the application will discuss a solvent comprising a ketone, however it is not desired to so limit the application.

The foundation layer of the gel nail polish comprises a solution of ethyl acetate, ammonium hydroxide, and a ketone, wherein the foundation layer is configured to remove built up oils and dirt accumulation from the nail surface, such that the nail is prepared to receive additional layers thereon. The foundation layer is configured to evaporate to leave a coating of the ketone on the nail surface, wherein the ketone is configured to penetrate through and react with additional layers of the gel nail polish system to cure each layer of the gel nail polish and adhere the gel nail polish to the nail surface. Furthermore, the foundation layer is configured to remove oil and dirt buildup accumulated on the nail surface, so as to prepare the nail surface for the application of further layers. The application of the foundation layer further serves to increase adhesion of additional layers applied after the foundation layer. The foundation layer is configured to serve as an activator for chemical reactions occurring between further layers, wherein the chemical reaction that occurs serves to cure the nail polish system. The ketone residue remaining on the nail surface is configured to permeate through each additional layer applied, the ketone reacting with each layer to cure the applied layers. One or more layers of the foundation layer can be applied to the nail to reach a thickness desired by the user. Additional layers further increase the concentration of the ketone on the nail surface. This allows multiple base, color, and top coat layers to be applied as additional reactant is available on the nail surface. In some embodiments, the foundation layer comprises 80% ethyl acetate, 15% ammonium hydroxide, and 5% ketone.

In one embodiment, the base layer comprises a solution of ethyl cyanoacrylate and the solvent, however alternate solutions are contemplated, including a solution of methyl methacrylate. In some embodiments, the solution comprises 50-100% ethyl cyanoacrylate. At least one base layer is applied to the nail surface after the foundation layer, wherein the ketone residue on the nail surface is configured to permeate through the base layer and chemically react therewith to cure the base layer. In this sense, curing is defined as the toughening or hardening of a polymer material via cross-linking of polymer chains, in this case, via chemical processes. In traditional gel nail polish systems, ultraviolet radiation is required to cure the system. Should a user wish to apply multiple base layers, a number of foundation layers equal to the desired number of base layers must be applied in order to ensure that enough ketone activator is present on the nail to fully cure each base layer. The base layer is configured to fill in any uneven portions of the nail surface in order to provide a smooth surface, thereby ensuring that the final gel nail polish is smooth and even. The base layer further serves to protect the nail from any damage caused by nail polish application, such as staining the nail with pigment.

The color layer comprises a solution of ethyl methacrylate, a ketone, a plasticizer, and a pigment, wherein the ketone is configured to penetrate through adjacent layers to allow the color layer to cure to the adjacent layers. In some embodiments, the pigment comprises 5% of the color layer, and the plasticizer comprises 25% of the color layer. The plasticizer is configured to facilitate superior adhesion, cure into a hardened layer when reacted with, and allows the gel nail polish system to easily be removed when a solvent configured to depolymerize the gel nail polish solution is applied. In one embodiment, the plasticizer comprises methacrylate polymer, however alternative plasticizers are contemplated, including, but not limited to cellulose acetate, cellulose acetate butyrate, ethyl cellulose, polyesters, resins, epoxies, methacrylic and vinyl copolymers, ethylene acetate, vinyl acetate copolymers, and dimethicone.

The pigment is configured to provide a color to the color layer, allowing a user to select a desired color for personal aesthetic preferences. The pigment can comprise a mixture of one or more of the following pigments: cadmiums, titanium dioxide, barium, calcium and aluminum lakes, bismuth oxychlorides, talcs, carmine, chromates, iron oxides, metallic or mixed metallic oxides, D&C Violet #2, D&C Green #5, D&C Red #6, D&C Red #7, D&C Red #17, D&C Red #21, D&C Red #22, D&C Red #27, D&C Red #28, D&C Red #30, D&C Red #31, D&C Red #33, D&C Red #34, D&C Red #36, D&C Red #40, FD&C Blue#1, D&C Yellow #5, D&C Yellow #7, D&C Yellow #8, D&C Yellow #10, D&C Yellow #11, FD&C Blue #1, FD&C Red #40, FD&C Red #4, FD&C Yellow #5, FD&C Yellow #6, D&C Black #2, D&C Orange #4, D&C Orange #5, D&C Orange #10, D&C Orange #11. However, this list is not exhaustive and alternate pigment options that are known to be inert in gel nail polish systems are also contemplated.

The top coat comprises a solution similar to the base coat, wherein the solution comprises ethyl cyanoacrylate. In some embodiments, the solution comprises 50-100% ethyl cyanoacrylate. The ketone from the foundation layer penetrates through each previous layer to react with the top coat, allowing the top coat to cure into a hard, durable, and scratch resistant surface. The top coat is configured to prevent damage to previous layers of the gel nail polish system, ensuring that the nail polish remains even and unblemished. In some embodiments, the top coat can be immediately buffed to a desired level of shine and polish after the top coat cures, as opposed to traditional nail polish which is prone to chipping, scratching, or other damage if buffed within days of application.

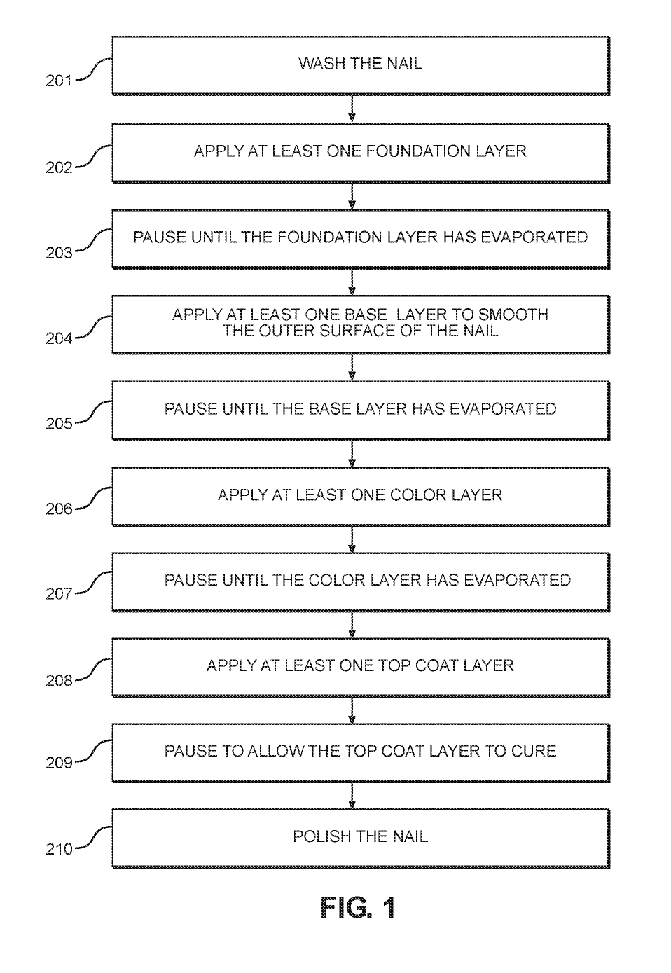

Referring now to FIG. 1, there is shown a flowchart of the method of applying an embodiment of the gel nail polish system. In one exemplary use, the user can first wash 201 the nail, such that the nail surface is properly prepared to receive the gel nail polish system thereon. The user can then apply at least one foundation layer 202, wherein the foundation layer is configured to leave a coating of ketone on the nail surface. The user can then pause until the foundation layer has evaporated 203, leaving a coating of ketone on the nail surface for reacting with later applied layers. In some embodiments, the foundation layer evaporates within one minute of application. The user can then apply at least one base layer 204, wherein the base layer is configured to fill in gaps and provide a smooth and even nail surface for the application of additional layers. The user can then pause until the base layer has evaporated 205, such that the ketone of the foundation layer reacts with the base layer to assist in the curing thereof. In some embodiments, the base layer fully evaporates within one minute of application. The user can then optionally choose to apply at least one color layer 206, so as to apply an aesthetic colorful appearance to the nail, however this step can be skipped should a user desire to have a natural nail color protected by the gel nail polish system. The user then pauses to allow the color layer has evaporated 207 thereby curing the color layer. In some embodiments, the color layer evaporates within one minute of application. A top coat layer is then applied 208, wherein the top coat is configured to protect the nail surface from damage once cured. The user then pauses to allow the top coat to cure 209, ensuring that damage to the underlying layers is minimized thereby. In some embodiments, the top coat fully cures within two minutes of application. In this way, the entire process can be completed within five minutes. Finally, should the user desire to achieve a level of shine or smoothness, the user can polish the nail 210, wherein the nail can be buffed or polished to produce the desired aesthetic effect. As the gel nail polish system is self-curing and non-reactive to UV light like traditional gel nail polish, the self-curing gel nail polish can then be stored in transparent or translucent bottles without reacting to light passing therethrough, such that a user can visually inspect the color or pigment of the specific nail polish before purchase.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A gel nail polish system, comprising:
a foundation layer comprising a solution of ethyl acetate, ammonium hydroxide, and a ketone;
wherein the foundation layer is configured to remove oil and dirt buildup on a nail and evaporate over time leaving a deposit of ketone on the nail;
a base layer comprising a solution of ethyl cyanoacrylate;
wherein the base layer is configured to provide a uniform surface across the nail;
a top coat comprising a solution of ethyl cyanoacrylate;
wherein the top coat is configured to harden to protect the base and foundation layers from damage;
wherein each of the foundation layer, base layer, and top coat are in solution with a solvent.

2) The gel nail polish system of claim 1, further comprising a color layer a solution of ethyl methacrylate, a ketone, a plasticizer, and a pigment, wherein the ketone is configured to penetrate through the base layer to react with the foundation layer to harden each applied layer.

3) The gel nail polish system of claim 1, wherein the foundation layer comprises 80% ethyl acetate, 15% ammonium hydroxide, and 5% ketone.

4) The gel nail polish system of claim 1, wherein the base layer comprises 50-100% ethyl cyanoacrylate, wherein the balance comprises the solvent.

5) The gel nail polish system of claim 2, wherein the pigment comprises 5% of the color layer.

6) The gel nail polish system of claim 1, wherein the solvent comprises one or more compounds selected from a group consisting essentially of ketones, butyl acetate, ethyl acetate, propyl acetate, dimethyl-p-toluidine, N-dimethyl-p-toluidine, butyl alcohol, isopropyl alcohol, ethanol, amyl acetate, or acetone.

7) The gel nail polish system of claim 2, wherein the plasticizer comprises one or more compounds selected from a group consisting essentially of methacrylate polymer, cellulose acetate, cellulose acetate butyrate, ethyl cellulose, polyester, resin, epoxy, methacrylate copolymers, vinyl copolymers, ethylene acetate, vinyl acetate copolymers, and dimethicone.

8) The gel nail polish system of claim 2, wherein the pigment comprises one or more compounds selected from a group consisting essentially of cadmium, titanium dioxide, barium, calcium lake, aluminum lake, bismuth oxychlorides, talcs, carmine, chromates, iron oxide, metallic oxides, D&C Violet #2, D&C Green #5, D&C Red #6, D&C Red #7, D&C Red #17, D&C Red #21, D&C Red #22, D&C Red #27, D&C Red #28, D&C Red #30, D&C Red #31, D&C Red #33, D&C Red #34, D&C Red #36, D&C Red #40, D&C Blue #1, D&C Yellow #5, D&C Yellow #7, D&C Yellow #8, D&C Yellow #10, D&C Yellow #11, FD&C Blue #1, FD&C Red #40, FD&C Red #4, FD&C Yellow #5, FD&C Yellow #6, D&C Black #2, D&C Orange #4, D&C Orange #5, D&C Orange #10, and D&C Orange #11.

9) A method of applying a gel nail polish system, comprising:
applying at least one foundation layer comprising a solution of ethyl acetate, ammonium hydroxide, and a ketone to remove dirt and oil buildup on a nail;
pausing until the foundation layer has fully evaporated leaving a coating of ketone on the nail;
applying at least one base layer comprising a solution of ethyl cyanoacrylate, wherein the base layer is configured to smooth an outer surface of the nail such that the nail comprises a uniform outer surface;
pausing until the base layer has fully evaporated to allow the ketone to penetrate through each of the layers and react to harden each of the applied layers;
applying at least one top coat layer comprising a solution of ethyl cyanoacrylate;
pausing to allow the top coat to cure to protect each of the previously applied layers.

10) The method of applying a gel nail polish system of claim 8, further comprising washing the nail before applying the foundation layer.

11) The method of applying a gel nail polish system of claim 8, further comprising applying a color layer before applying the top coat, wherein the color layer comprises a solution of ethyl methacrylate, a ketone, a plasticizer, and a pigment.

12) The method of applying a gel nail polish system of claim 10, further comprising pausing until the color layer has evaporated, such that the ketone therein has reacted with the previously applied layers.

13) The method of applying a gel nail polish system of claim 8, further comprising polishing the nail after the top coat has fully cured.
